(19) United States
(12) Patent Application Publication
(10) Pub. No.: US 2009/0056734 A1
(43) Pub. Date: Mar. 5, 2009

(54) Hair texturizer and conditioner

(76) Inventor: Rebecca Bacon, Los Angeles, CA (US)

Correspondence Address: Cislo & Thomas LLP, 1333 2nd Street, Suite #500, Santa Monica, CA, 90401-4110, US

(21) Appl. No.: 11/897,670

(22) Filed: Aug. 31, 2007

(51) Int. Cl. A61K 8/92; A61K 8/34; A61K 8/97; A61Q 5/06; A61Q 5/12

(52) U.S. Cl. 132/202; 424/70.1; 424/74

(57) ABSTRACT

The present invention relates to personal care compositions, and in particular, a hair care composition for obtaining tangled and tousled hair styles. The compositions are preferably made of a large proportion of natural products and a minimal amount of drying agents and preservatives. In particular, the invention relates to a hair care composition comprising water, sea salt, coconut oil, panthenol, and SD alcohol. The invention further relates to methods of using the composition for obtaining fashionable tangled hair styles and methods of making the composition that are economical and easy to manufacture.

### FIELD OF THE INVENTION

This invention relates to personal care hair care compositions for emulating tousled and tangled hair styles and methods of making and using the compositions.

### BACKGROUND OF THE INVENTION

A day at the beach can create untame, tousled, and voluminous hair. While such hair may seem unfavorable to some, achieving the look can be fashionable and desired by many. Salt, sand, and wind aid in creating the tousled, fashionable look. Exposure to these elements, however, can be damaging to hair. Dry hair is problematic because it makes hair weak and frizzy and causes unsightly split ends. Furthermore, achieving the look of having spent a day at the beach may not be possible for those that do not have access to such conditions.

Accordingly, achieving the “beach ready” hair look without having to spend a day at the beach has been attempted by application of various hair care products to the hair. Many such products, however, contain harmful ingredients that exacerbate the dry hair problem and may cause long term damage to hair. For example, many products contain large amounts of drying agents and lack protecting agents. Many products may also contain large portions of synthetic chemicals that are harmful to the user and may cause long-term ill health effects. Furthermore, many compositions require complex manufacturing processes thus increasing the cost of production.

It is a goal of the present invention to provide a hair care composition that does not dry out the hair, but that provides the fashionable tangled, tousled look of one who spent the day at the beach. The composition is preferably comprised of a large proportion of organic, natural ingredients. An economical method of making the composition and a method of using the composition to achieve a day-at-the-beach look for hair is also contemplated.

### SUMMARY OF THE INVENTION

The present invention relates to a composition for the hair for creating tangled, tousled hair styles, also called “beach-ready” hair, comprising a vehicle, one or more texturizers, a shine enhancer, a fragrance, a protecting agent, a preservative, and optionally a coloring agent. In a preferred embodiment, the composition comprises a vehicle such as water, a texturizer such as sea salt, a shine enhancer and fragrance such as coconut oil (which can also function as a conditioner and texturizer), a protecting agent such as panthenol, a preservative such as specially denatured alcohol or “SD alcohol,” and optionally a coloring agent such as natural orange food coloring. Additional fragrance may be added. Other ingredients performing the aforementioned functions are contemplated.

The present invention also relates to a method of making the composition comprising boiling water, adding sea salt and simmering until dissolved, adding coconut oil and boiling until dissolved, cooling, removing and discarding any solid material in the cooled mixture by skimming and/or straining, and adding panthenol, alcohol, and an optional coloring agent to the remaining mixture. Additional fragrance may also be added to the mixture.

A method of using the composition for achieving untamed hair styles is contemplated comprising shaking the composition, spraying or spritzing it onto the hair, and styling as desired. The hair may be wet or dry.

### DETAILED DESCRIPTION

The detailed description set forth below is intended as a description of exemplary embodiments and is not intended to represent the only forms in which these embodiments may be formulated and/or utilized. The description sets forth the functions and the sequence of steps for preparing and using the exemplary embodiments. It is to be understood, however, that the same or equivalent functions and sequences of steps for preparation and use may be accomplished by different embodiments that are encompassed within the spirit and scope of the specification. For example, the sample methods of making the composition provided may be scaled up or down depending upon the amount of composition desired. Alternatively, ingredients having the same functions as those ingredients noted herein may be substituted for the listed ingredients.

A hair care composition is contemplated comprising a vehicle, one or more texturizers, a shine enhancer and fragrance, a protecting agent, a preservative, and optionally a coloring agent. Additional ingredients such as fragrance may also be added.

The vehicle may be water; the texturizer may be sea salt; the shine enhancer and fragrance may be coconut oil (which also conditions and texturizes hair); the protecting agent may be panthenol; the preservative may be SD alcohol; and the optional coloring agent may be natural orange food coloring.

In another embodiment, the vehicle may be selected from the group consisting of water, SD alcohol, and mixtures thereof.

The protecting agents may be selected from the group consisting of panthenol, dexpanthenol, vitamin-B complex factor, provitamin B-5, lanolin, and mixtures thereof.

The shine enhancers may be selected from the group consisting of coconut oil, shea butter, almond oil, olive oil, chamomile oil, palm oil, rosemary extract, silicone, derivatives of silicone, henna, aloe vera, fruit extracts, green tea extract, jojoba, lemon juice, vinegar, and mixtures thereof.

The preservatives may be selected from the group consisting of SD alcohol, one or more of the essential oils such as thyme, organum, sweet orange, lemongrass, Chinese cinnamon, rose, clove, eucalyptus, peppermint, or rose geranium, and mixtures thereof.

The fragrances may be selected from the group consisting of coconut oil, fragrance, coumarin, hydroxyisohexyl 3-cyclohexene, carboxaldehyde, alpha-isomethyl ionone, butylphenyl methylpropional, limonene, linalool, geraniol, citral; butylmethoxydibenzoylmethane, ethylhexyl methoxycinnamate, ethylhexyI salicylate, and mixtures thereof.

In one embodiment, the composition comprises 70 to 90% vehicle, 5 to 20% texturizer(s), 0.5 to 10% shine enhancer and fragrance, 0.1 to 5.0% protecting agent, 0.05 to 5.0% preservative, and optionally 0.025 to 2.5% coloring agent, or equivalent proportions therein.

In a preferred embodiment, the composition comprises 80 to 90% vehicle, 7.5 to 15% texturizer(s), 1 to 5% shine enhancer and fragrance, 0.5 to 2.5% protecting agent, 0.5 to 1.5% preservative, and optionally 0.25 to 1.0% coloring agent, or equivalent proportions therein.

In a further preferred embodiment, the composition comprises in approximate percentages 82.4% vehicle, 12.4% texturizer(s), 1.95% shine enhancer and fragrance, 1.85% protecting agent, 0.9% preservative, and optionally 0.50% coloring agent.

In yet another embodiment, the composition comprises 17 to 22 L vehicle, 1.2 to 5.0 L texturizer(s), 0.12 to 2.50 L shine enhancer and fragrance, 0.02 to 1.30 L protecting agent, 0.01 to 1.30 L preservative, and optionally 0.006 to 0.60 L coloring agent, or equivalent proportions therein. For example, the composition may comprise 17 to 22 L water, 1.2 to 5.0 L sea salt, 0.12 to 2.50 L coconut oil, 0.02 to 1.30 L panthenol, 0.01 to 1.30 L SD alcohol, and optionally 0.006 to 0.60 L coloring agent, or equivalent proportions therein.

In another embodiment, the composition comprises 19 to 22 L vehicle, 1.8 to 3.7 L texturizer(s), 0.24 to 1.30 L shine enhancer and fragrance, 0.1 to 0.6 L protecting agent, 0.1 to 0.4 L preservative, and optionally 0.06 to 0.25 L coloring agent, or equivalent proportions therein.

In a further preferred embodiment, the composition comprises in approximate volumes, 20 L vehicle, 3 L texturizer(s), 0.473 L shine enhancer and fragrance, 0.459 protecting agent, 0.230 L preservative, and optionally 0.115 L coloring agent, or equivalent proportions therein.

In all of the aforementioned embodiments, additional fragrance may be added.

A method of making the composition is further contemplated comprising boiling water, adding sea salt until dissolved, adding coconut oil and bringing the composition to a boil, cooling the composition by removing from heat and/or refrigerating, removing and discarding solid material in the mixture by skimming and/or straining, and adding panthenol, alcohol, and an optional coloring is agent to the remaining mixture. The method may further comprise adding additional fragrance to the remaining mixture. The mixture may be poured into one or more small spray bottles or other containers either before or after adding panthenol, alcohol, and an optional coloring agent.

### EXAMPLE 1

The hair care composition is made by bringing 20 liters of water to a boil. Three liters (or 12.68 cups) of sea salt is slowly added to the water, stirring constantly for 7-10 minutes. The temperature is then reduced so that the composition simmers. The composition simmers for approximately 10-15 minutes, stirring occasionally until all of the sea salt is dissolved. Once the sea salt is dissolved, two cups (or 0.473 liters) of organic coconut oil are added and the composition is brought to a boil. The composition is boiled for approximately 10 minutes or until the coconut oil is dissolved. Next, the heat is turned off, and the composition is cooled for about an hour. The composition is then placed in the refrigerator and cooled for at least 5 hours, or overnight. After cooling in the refrigerator, the solid, hardened layer that forms on the top of the composition is removed. Next, the contents are poured through a straining medium such as cheese cloth into a large, preferably a 22 quart, container. The straining medium is intended to filter out any remaining hardened pieces in the mixture. Alternatively, the hardened pieces may be skimmed from the mixture. Then, small bottles, preferably 2 ounce bottles, are substantially filled with the composition and ¼ teaspoon of panthenol (approximately 0.04 fluid ounces), 2 drops of SD alcohol (approximately 0.02 fluid ounces), and optionally 1 drop of 100% natural orange food coloring is added. The composition is sealed in the bottle and can be shaken prior to using.

### EXAMPLE 2

The hair care composition is made by bringing 20 liters of water to a boil. Three liters (or 12.68 cups) of sea salt is slowly added to the water, stirring constantly for 7-10 minutes. The temperature is then reduced so that the composition simmers. The composition simmers for approximately 10-15 minutes, stirring occasionally until all of the sea salt is dissolved. Once the sea salt is dissolved, two cups (or 0.473; liters) of organic coconut oil are added and the composition is brought to a boil. The composition is boiled for approximately 10 minutes or until the coconut oil is dissolved. Next the heat is turned off, and the composition is cooled for about an hour. The composition is then placed in the refrigerator and cooled for at least 5 hours, or overnight. After cooling in the refrigerator, the solid, hardened layer that forms on the top of the composition is removed. Next, the contents are poured through a straining medium such as cheese cloth into a large, preferably a 22 quart, container. The straining medium is intended to filter out any remaining hardened pieces in the mixture. Alternatively, the hardened pieces may be skimmed from the mixture. Then, the following three ingredients are added to the mixture: 0.459 liters of panthenol, 0.230 liters of SD alcohol, and optionally 0.115 liters of 100% natural orange food coloring, and the resultant contents are mixed. Next, the composition is poured into bottles, preferably 2 ounce bottles. The composition is sealed in the bottle and can be shaken prior to using.

A method of using the composition to achieve an untamed, tousled hair style is also contemplated comprising shaking the composition and then spraying or spritzing the composition on dry or wet hair particularly the roots (and ends if blow drying is used for styling), and optionally styling the hair as desired. Styling the hair as desired may include styling using the hands, blow drying, using a curling iron or other heating device, or pinning the hair up in a hair ornament, pony tail elastic, clip, pin, or the like.

In particular a method of using the composition comprises spraying or spritzing on roots before blow drying to add volume; spraying or spritzing on ends after blow drying for individualized texture that gives a “flick” to the ends of the hair; spraying or spritzing on the roots before drying and before styling in an up-do to give expansion and texture to the hair; or spraying or spritzing on towel-dried hair and sculpting and letting air dry for a piecey texture and/or for creating air dried curls. The method may further comprise spritzing or spraying on hair throughout the day or a day after application to reactivate and volumize any style.

What is claimed is:

1. A hair care composition comprising water, sea salt, coconut oil, panthenol, and SD alcohol.

2. The composition of claim 1 wherein the composition comprises 17 to 22 L water, 1.2 to 5.0 L sea salt, 0.12 to 2.50 L coconut oil, 0.02 to 1.30 L panthenol, 0.01 to 1.30 L SD alcohol.

3. The composition of claim 1 further comprising a fragrance.

4. The composition of claim 1 further comprising a coloring agent.

5. The composition of claim 4 further comprising a fragrance.

6. The composition of claim 4 wherein the composition comprises 17 to 22 L water, 1.2 to 5.0 L sea salt, 0.12 to 2.5 L coconut oil, 0.02 to 1.30 L panthenol, 0.01 to 1.30 L SD alcohol, and 0.006 to 0.60 L coloring agent.

7. A method of making the composition of claim 1 comprising:
boiling water,
adding sea salt to the water to create a mixture and simmering the mixture until dissolved,
adding coconut oil to the mixture and boiling the mixture until the coconut oil is dissolved,
cooling the mixture to allow it to partially solidify,
removing and discarding the solid from the mixture, and
adding panthenol and SD alcohol to the mixture.

8. The method of claim 7 further comprising pouring the mixture into one or more containers.

9. The method of claim 7 further comprising adding a coloring agent to the mixture.

10. A method of making a hair care composition comprising:
boiling water,
adding sea salt to the water to create a mixture and simmering until dissolved,
adding coconut oil to the mixture and boiling the mixture until the coconut oil is dissolved,
cooling the mixture to allow it to partially solidify,
removing and discarding the solid from the mixture,
pouring the mixture into one or more containers, and
adding panthenol and SD alcohol each of the containers.

11. The method of claim 10 further comprising adding a coloring agent to each of the containers.

12. A method of using the composition of claim 1 comprising
shaking the composition,
applying the composition to hair by spraying or spritzing, and
styling the hair as desired.

13. A hair care composition comprising a vehicle, a texturizer, a shine enhancer, a fragrance, a protecting agent, and a preservative.

14. The composition of claim 13, wherein the vehicle is selected from the group consisting of water, SD alcohol, and mixtures thereof.

15. The composition of claim 13, wherein the texturizer is sea salt.

16. The composition of claim 13, wherein the shine enhancer is selected from the group consisting of coconut oil, shea butter, almond oil, olive oil, chamomile oil, palm oil, rosemary extract, silicone, derivatives of silicone, henna, aloe vera, fruit extracts, green tea extract, jojoba, lemon juice, vinegar, and mixtures thereof.

17. The composition of claim 13, wherein the fragrance is selected from the group consisting of coconut oil, fragrance, coumarin, hydroxyisohexyl 3-cyclohexene, carboxaldehyde, alpha-isomethyl ionone, butylphenyl methylpropional, limonene, linalool, geraniol, citral; butylmethoxydibenzoylmethane, ethylhexyl methoxycinnamate, ethylhexyl salicylate, and mixtures thereof.

18. The composition of claim 13, wherein the protecting agent is selected from the group consisting of panthenol, dexpanthenol, vitamin-B complex factor, provitamin B-5, lanolin, and mixtures thereof.

19. The composition of claim 13, wherein the preservative is selected from the group consisting of alcohol SD alcohol, one or more essential oils, and mixtures thereof.

20. The composition of claim 13, further comprising a coloring agent.
